(19) United States
(12) Patent Application Publication
(10) Pub. No.: US 2009/0312286 A1
(43) Pub. Date: Dec. 17, 2009

(54) DERIVATIVES OF 4-TRIMETHYLAMMONIUM-3-AMINOBUTYRATE AND 4-TRIMETHYLPHOSPHONIUM-3-AMINOBUTYRATE AS CPT-INHIBITORS

(75) Inventors: Fabio Giannessi, Pomezia (IT); Emanuela Tassoni, Ciampino (IT); Natalina Dell' Uomo, Pomezia (IT); Roberto Conti, Roma (IT); Maria Ornella Tinti, Roma (IT)

Correspondence Address: LUCAS & MERCANTI, LLP, 475 PARK AVENUE SOUTH, 15TH FLOOR, NEW YORK, NY, 10016, US

(73) Assignee: SIGMA-TAU INDUSTRIE FARMACEUTICHE RIUNITE S.P.A., Roma (IT)

(21) Appl. No.: 12/374,774

(22) PCT Filed: Jul. 10, 2007
(86) PCT No.: PCT/EP07/57030

§ 371 Date: Mar. 11, 2009

(30) Foreign Application Priority Data
Aug. 2, 2006 (EP) 06118338.0

(51) Int. Cl. A61K 31/195; C07C 273/00; C07C 229/34; A61P 3/10; A61P 9/00; A61K 31/66

(52) U.S. Cl. 514/77; 562/439; 562/448; 514/564; 514/563

(57) ABSTRACT

The invention provides a new class of compounds capable of inhibiting carnitine palmitoyl transferase (CPT) having formula (I)

The invention also relates to pharmaceutical compositions, which comprise at least one new compound according to the invention, and their therapeutic use in the treatment of hyperglycaemic conditions such as diabetes and the pathologies associated with it, such as for example congestive heart failure and obesity.

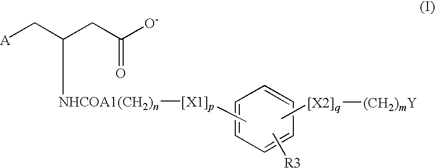

### FIELD OF THE INVENTION

The present invention describes a new class of compounds capable of inhibiting carnitine palmitoyl transferase (CPT); the invention also relates to pharmaceutical compositions, which comprise at least one new compound according to the invention, and their therapeutic use in the treatment of hyperglycaemic conditions such as diabetes and the pathologies associated with it, such as for example congestive heart failure and obesity.

### BACKGROUND OF THE INVENTION

Known hypoglycaemic treatment is based on the use of drugs with a different mechanism of action (Arch. Intern. Med. 1997, 157, 1802-1817).

The more common treatment is based on insulin or its analogues, which uses the direct hypoglycaemic action of this hormone.

Other compounds act indirectly by stimulating the release of insulin (sulfonyl ureas). Another target of the hypoglycaemic drugs is the reduction of the intestinal absorption of glucose via the inhibition of the intestinal glucosidases, or the reduction of insulin resistance. Hyperglycaemia is also treated with inhibitors of gluconeogenesis such as the biguanides.

Some authors have shown the relationship between gluconeogenesis and the enzyme carnitine palmitoyl transferase.

Carnitine palmitoyl transferase catalyses the formation in the cytoplasm of palmitoyl carnitine (activated fatty acid) from carnitine and palmitoyl coenzyme A. Palmitoyl carnitine is different from palmitic acid in that it easily crosses the mitochondrial membrane. Palmitoyl coenzyme A reconstitutes itself within the mitochondrial matrix, releasing carnitine. Palmitoyl coenzyme A is oxidised to acetyl-coenzyme A, which activates pyruvic carboxylase, a key enzyme in the gluconeogenic pathway.

Some authors report that diabetic patients have high blood levels of fatty acids which are oxidised in the liver producing acetylcoenzyme A, ATP and NADH. The high availability of these substances causes over-regulation of gluconeogenesis, with a subsequent increase in the level of blood glucose. In these situations, the inhibition of CPT would limit the oxidation of the fatty acids and then, consequently, gluconeogenesis and hyperglycaemia. Inhibitors of CPT have been described in J. Med. Chem., 1995, 38(18), p. 3448-50, and in the relevant European patent application EP-A-574355 as potential derivatives with hypoglycaemic action.

The international patent application WO99/59957 in the name of the Applicant describes and claims a class of derivatives of butyric acid which have displayed inhibitory action on CPT. An example of these compounds is R-4-trimethyl ammonium-3-(tetradecyl carbamoyl)-aminobutyrate (ST1326).

It has recently been demonstrated that the inhibition of CPT-1 in the hypothalamus, produced experimentally by administering intracerebroventricular inhibitors (icv), is capable of significantly and consistently reducing, in terms of extent and duration of the effect, food intake and gluconeogenesis (Nature Medicine, 2003, 9(6), 756-761). This property has also been demonstrated using the compound ST1326.

It is always an object of the researchers to find compounds having increased efficacy especially when administered by oral route.

### DESCRIPTION OF THE INVENTION

The present invention relates to new inhibitors of carnitine palmitoyl transferase I with the following formula (I):

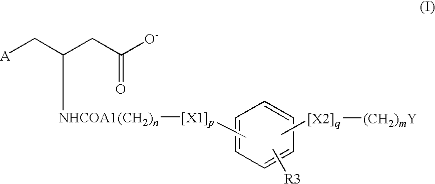

wherein:
A is selected between —N⁺ (R R₁, R₂), —P⁺ (R R₁, R₂), in which R, R₁, R₂ are the same or different and are selected from the group consisting of (C₁-C₂) alkyl, phenyl and phenyl-(C₁-C₂) alkyl; A1 is O or NH or is absent;
n is an integer number ranging from 0 to 20;
p is 0 or 1; q is 0, 1;

### X1 is O or S;

### X2 is O or S;

m is an integer number ranging from 1 to 20;
Y selected among H, phenyl and phenoxy;
R3 is selected among H, halogen, linear or branched (C₁-C₄) alkyl and (C₁-C₄) alkoxy.

Preferably R, R₁ and R₂ are all methyl. Preferably m is an integer number ranging from 1 to 10, more preferably from 4 to 8.

For the purposes of the present invention it is clarified that each of the products of formula (I) can exist both as a racemic mixture R/S, and in the separate isomeric forms R and S.

The present invention also comprises tautomers, geometrical isomers, optically active forms as enantiomers, diastereomers and racemate forms, as well as pharmaceutically acceptable salts of the compounds of Formula (I). The present invention covers all these different possibilities of salification for the compounds of formula (I).

Preferred pharmaceutically acceptable salts of the Formula (I) are acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

Within the framework of the present invention, examples of the linear or branched (C₁-C₄) alkyl group, are understood to include methyl, ethyl, propyl and butyl and their possible isomers, such as, for example, isopropyl, isobutyl, and ter-butyl.

The following are some of the most preferred compounds according to the invention:
(R)-4-trimethylammonium-3-[[4-[(3-hexyloxy)-phenoxy]butyl]carbamoyl]-amino-butyrate (ST2425);
(R)-4-trimethylphosphonium-3-[[4-[(3-hexyloxy)-phenoxy]buty]carbamoyl]-amino-butyrate (ST2452);
(R)-4-trimethylammonium-3-[[4-(heptyloxy)-phenyl]-carbamoyl]-amino-butyrate (ST2773);
(R)-4-trimethylammonium-3-[[2-(benzyloxy)-benzyl]carbamoyl]-amino-butyrate (ST2790);
(R)-4-trimethylammonium-3-[[(4-benzyloxy-3-methoxy)-benzyl]carbamoyl]-amino-butyrate (ST2816);
(R)-4-trimethylammonium-3-[[4-[(2-hexyloxy)-phenoxy]butyl]carbamoyl]-amino-butyrate (ST4005);
(R)-4-trimethylammonium-3-[[4-[(3-hexyloxy)-phenoxy]propil]carbamoyl]-amino-butyrate (ST4024); and
(R)-4-trimethylammonium-3-[[3-(hexyloxy)phenoxy]acetyl]-amino-butyrate (ST4004).

A further object of the invention described herein are compounds with general Formula (I) for use in the medical field.

A further object of the invention described herein is a pharmaceutical composition containing as active ingredient a compound of Formula (I) and at least a pharmaceutically acceptable excipient and/or diluent.

The compounds of formula (I) have inhibitory activity on carnitine palmitoyl transferases. This activity makes it possible to use them in the treatment and/or in the prevention of obesity, hyperglycaemia, diabetes and associated disorders such as, for example, diabetic retinopathy, diabetic neuropathy and cardiovascular disorders. The compounds of formula (I) are also used in the prevention and treatment of cardiac disorders such as congestive heart failure.

The inhibitory action of the compounds of formula (I) takes place mainly on isoform 1 of carnitine palmitoyl transferase (CPT-1) and, in particular, also in the hypothalamus.

A further object of the invention described herein is a pharmaceutical composition containing as active ingredient a compound Formula (I), for the treatment and/or in the prevention of obesity, hyperglycaemia, diabetes and associated disorders such as, for example, diabetic retinopathy, diabetic neuropathy and cardiovascular disorders. The compounds of formula (I) are also used in the prevention and treatment of cardiac disorders such as congestive heart failure.

Another object of the present invention is a process for preparing any of the pharmaceutical compositions as mentioned above, comprising mixing the compound(s) of Formula (I) with suitable excipient and/or diluent.

A further object of the invention described herein is the use of a compound of Formula (I) for the preparation of a medicine for the treatment and/or in the prevention of obesity, hyperglycaemia, diabetes and associated disorders such as, for example, diabetic retinopathy, diabetic neuropathy and cardiovascular disorders. The compounds of formula (I) are also used in the prevention and treatment of cardiac disorders such as congestive heart failure.

Another object of the invention is a method of treating a mammal suffering from obesity, hyperglycaemia, diabetes and associated disorders, comprising administering a therapeutically effective amount of the compound(s) of Formula (I).

“Therapeutically effective amount” is an amount effective to achieve the medically desirable result in the treated subject. The pharmaceutical compositions may contain suitable pharmaceutical acceptable carriers, biologically compatible vehicles suitable for administration to an animal (for example, physiological saline) and optionally comprising auxiliaries (like excipients, stabilizers or diluents) which facilitate the processing of the active compounds into preparations which can be used pharmaceutical.

For any compound, the therapeutically effective dose can be estimated initially either in cell culture assays or in animal models, usually mice, rats, guinea pigs, rabbits, dogs, or pigs.

The animal model may also be used to determine the appropriate concentration range and route of administration. Such information can then be used to determine useful doses and routes for administration in humans.

The pharmaceutical compositions may be formulated in any acceptable way to meet the needs of the mode of administration. The use of biomaterials and other polymers for drug delivery, as well the different techniques and models to validate a specific mode of administration, are disclosed in literature.

Modifications of the compounds of the invention to improve penetration of the blood-brain barrier would also be useful.

Any accepted mode of administration can be used and determined by those skilled in the art. For example, administration may be by various parenteral routes such as subcutaneous, intravenous, intradermal, intramuscular, intraperitoneal, intranasal, transdermal, oral, or buccal routes.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyloleate or triglycerides.

Aqueous injection suspensions that may contain substances increasing the viscosity of the suspension include, for example, sodium carboxymethyl cellulose, sorbitol, and/or dextran. Optionally, the suspension may also contain stabilizers.

Pharmaceutical compositions for intranasal administration may advantageously contain chitosan.

Pharmaceutical compositions include suitable solutions for administration by injection, and contain from about 0.01 to 99 percent, preferably from about 20 to 75 percent of active compound together with the excipient. Compositions which can be administered rectally include suppositories. It is understood that the dosage administered will be dependent upon the age, sex, health, and weight of the recipient, kind of concurrent treatment, if any, frequency of treatment, and the nature of the effect desired. The dosage will be tailored to the individual subject, as is understood and determinable by one of skill in the art. The total dose required for each treatment may be administered by multiple doses or in a single dose. The pharmaceutical composition of the present invention may be administered alone or in conjunction with other therapeutics directed to the condition, or directed to other symptoms of the condition. Usually a daily dosage of active ingredient is comprised between 0.01 to 100 preferably between 0.05 and 50 milligrams per kilogram of body weight.

The compounds of the present invention may be administered to the patient intravenously in a pharmaceutical acceptable carrier such as physiological saline.

Standard methods for intracellular delivery of peptides can be used, e.g. delivery via liposomes. Such methods are well known to those of ordinary skill in the art. The formulations of this invention are useful for parenteral administration, such as intravenous, subcutaneous, intramuscular and intraperitoneal.

As well known in the medical arts, dosages for any one patient depends upon many factors, including the patient's size, body surface area, age, the particular compound to be administered, sex, time and route of administration, general health, and other drugs being administered concurrently.

A further embodiment of the invention is a process for the preparation of pharmaceutical compositions characterised by mixing one or more compounds of formula (I) with suitable excipients, stabilizers and/or pharmaceutically acceptable diluents.

The compounds of Formula (I) may be prepared from readily available starting materials using the following general methods and procedures. It will be appreciated that where typical or preferred experimental conditions (i.e. reaction temperatures, time, moles of reagents, solvents, etc.) are given, other experimental conditions can also be used, unless otherwise stated. Optimum reaction conditions may vary with the particular reactants or solvents used, but such conditions can be determined by one skilled in the art by routine optimisation procedures.

A process for preparing the compounds of the present invention comprises reacting preferentially aminocarnitine and phosphoaminocarnitine with the corresponding isocyanates, in a dipolar aprotic or protic solvent, preferentially such as THF or MeOH, at temperatures comprised between 4° C. and the reflux temperature of the solvent, preferentially between 25 and 40° C., for times comprised between 1 and 72 hours, preferentially 24-48 hours. The isocyanates may be produced starting from the appropriate carboxylic acid via acylchloride and subsequent transformation into acylazide, or in situ using diphenylphosphorylazide.

The invention will now be illustrated in greater detail by means of non-limiting Examples, which will make reference to the following Figures.

### DESCRIPTION OF THE FIGURES

FIG. 1 shows the effect of oral administration of the new CPT I inhibitors of Formula (I) on ketone bodies production in fasted rats. The compounds were administered per os at 9:00 after 17 hours of fasting (n=5) at doses equimolar to 10 mg/kg of ST1326, which is used as reference compound.

FIG. 2 reports the dose-related effect of compound ST2425 on ketone bodies levels in fasted rats. A faster onset of action was also observed for this compound.

FIG. 3 reports the food intake (expressed as g/kg b.w.) in Sprague Dawley rats, treated intranasally for 3 days with ST2425 (320 μg/40 μl/rat) equally subdivided in the two nostrils. (Mean±S.D. (n=5). One way ANOVA post-hoc test SNK *p≦0.05 vs Control)

### EXAMPLES

### Example 1

### Preparation of (R)-4-trimethylammonium-3-[[4-(heptyloxy)phenyl]-carbamoyl]amino-butyrate (ST2773)

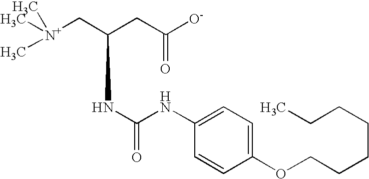

To a solution of (R)-aminocarnitine (149 mg, 0.93 mmol) in anhydrous MeOH (3.2 ml) at 5° C. 4-(heptyloxy)phenyl isocyanate (500 mg, 2.14 mmol) was added. The reaction mixture was stirred at room temperature for 48 hours, then the solid was filtered off. The solvent was evaporated under vacuum and the residue was triturated several times with diethyl ether and then desiccated under vacuum to give 200 mg of the desired product (55% yield). TLC: silica gel, R_{f}=0.49 (42:7:28:10.5:10.5 CHCl₃/isopropanol/MeOH/CH₃COOH/H₂O); [α]²⁰_{D}=−−21.50 (c=0.5%, MeOH); ¹H NMR (300 MHz, MeOH-d₄) δ 7.32 (d, 2H), 6.92 (d, 2H), 4.68 (br s, 1H), 4.01 (t, 2H), 3.83-3.58 (m, 2H), 3.31 (s, 9H), 2.58 (t, 2H), 1.86-1.79 (m, 2H), 1.58-1.43 (m, 8H), 1.03-0.98 (m, 3H); HPLC: column spherisorb SCX (5 μm-4.6×250 mm), mobile phase KH₂PO₄ 50 mM/CH₃CN 70/30 v/v, pH as it is, room temperature, flow rate 0.75 mL/min, detector UV 205 nm, retention time=6.7 min; K.F.=5.8% H₂O; A.E. in conformity with C₂₁H₃₅N₃O₄.

### Example 2

### Preparation of (R)-4-trimethylammonium-3-[[(4-benzyloxy-3-methoxy)-benzyl]carbamoyl]-amino-butyrate (ST2816)

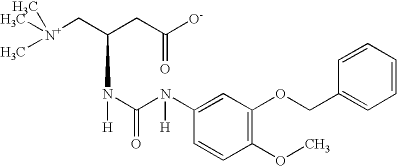

Triethylamine (357.3 μL, 2.57 mmol) was added to a solution of 4-benzyloxy-3-methoxyphenylacetic acid (700 mg, 2.75 mmol) in 7 ml of anhydrous THF, and the solution was stirred at room temperature for 30 minutes. Diphenylphosphorilazide (554 μL, 2.57 mmol) was then added and the solution was refluxed for 6 hours. The solution was chilled to 5-10° C. and added of a solution of (R)-aminocarnitine (206 mg, 1.28 mmol) in 3.5 mL of anhydrous methanol. The so obtained solution was stirred for 48 hours at room temperature, then the solvent was evaporated under vacuum and the residue was purified by flash chromatography on silica gel eluting by CH₃OH/AcOEt 9/1 giving 390 mg (60.6% yield) of product as a white solid. Mp 139-141° C.; TLC: silica gel, R_{f}=0.47 (42:7:28:10.5:10.5 CHCl₃/isopropanol/MeOH/CH₃COOH/H₂O); [α]²⁰_{D}=−16° (c=0.5%, MeOH); ¹H NMR (300 MHz, MeOH-d₄) δ 7.5 (d, 1H), 7.42 (m, 4H), 7.0 (m, 2H), 6.85 (dd, 1H), 5.15 (s, 2H), 4.60 (m, 1H), 4.30 (m, 2H), 3.90 (s, 3H), 3.70 (dd, 1H), 3.55 (dd, 1H), 3.25 (s, 9H), 2.51 (m, 2H); HPLC: column spherisorb S5 SCX (4.6×250 mm), mobile phase CH₃CN/KH₂PO₄ 50 mM 30/70 v/v, pH as it is, room temperature, flow rate=0.7 mL/min, detector UV 205 nm, retention time=7.4 min; K.F.=1.15% H₂O A.E. in conformity with C₂₃H₃₁N₃O₅.

### Example 3

### Preparation of (R)-4-trimethylammonium-3-[[2-(benzyloxy)-benzyl]carbamoyl]-amino-butyrate (ST2790)

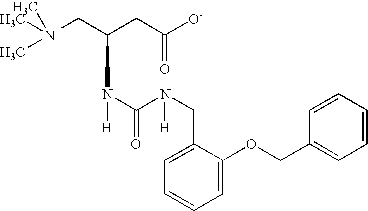

A solution of 2-benzyloxyphenylacetic acid (900 mg, 3.71 mmol) in 10 ml of anhydrous THF was added of triethylamine (516 μL, 3.71 mmol) and stirred at room temperature for 30 minutes. Diphenylphosphorilazide (796 μL, 3.71 mmol) was then added and the solution was refluxed for 6 hours. The solution was chilled to 5-10° C. and added of a solution of (R)-aminocarnitine (297 mg, 1.85 mmol) in 5 mL of anhydrous methanol. The so obtained solution was stirred for 48 hours at room temperature then the solvent was evaporated under vacuum and the residue was purified by flash chromatography on silica gel eluting by CH₃OH/AcOEt 9/1 giving 420 mg (56.7% yield) of product as a white solid. Mp 150-152° C.; TLC: silica gel, R_{f}=0.54 (42:7:28:10.5:10.5 CHCl₃/isopropanol/MeOH/CH₃COOH/H₂O); [α]²⁰_{D}=−20.5° (c=0.5%, MeOH); ¹H NMR (300 MHz, MeOH-d₄) δ 7.50-7.20 (m, 7H), 7.10 (d, 1H), 6.95 (t, 1H), 5.16 (s, 2H), 4.55 (m, 1H), 4.40 (dd, 2H), 3.65-3.45 (m, 2H), 3.15 (s, 9H), 2.45 (m, 2H); HPLC: column Spherisorb SCX (5 μm-4.6×250 mm), mobile phase CH₃CN/KH₂PO₄ 50 mM 30/70 v/v, pH as it is, room temperature, flow rate=0.7 mL/min, detector UV 205 nm, retention time=8.3 min; K.F.=1.81% H₂O, A.E. in conformity with C₂₂H₂₉N₃O₄.

### Example 4

### Preparation of (R)-4-trimethylammonium-3-[[4-[(3-hexyloxy)-phenoxy]butyl]carbamoyl]-amino-butyrate (ST2425)

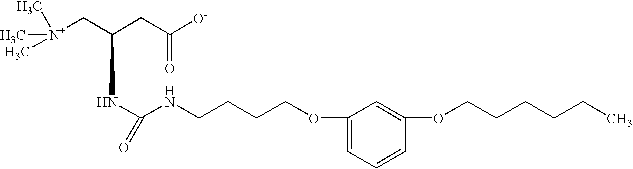

### Preparation of the Intermediate 3-hexyloxyphenol

The titled compound was prepared starting from resorcinol (4.00 g, 36.3 mmol) in 230 mL of anhydrous DMF and NaH (0.87 g, 36.3 mmol). The mixture was left under magnetic stirring for 20 minutes at room temperature, then 1-bromohexane (5.99 g, 36.3 mmol) was added. The reaction mixture was left 72 hours at 80° C. then was poured in H₂O (about 1 L) and extracted with AcOEt (3×250 mL). The organic layer was dried on Na₂SO₄, filtered, the solvent evaporated and the obtained residue (6.50 g, 97% yield) was used without further purification; ¹H NMR (CDCl₃, 300 MHz), δ 7.10 (brm, 1H), 6.50 (m, 3H), 3.98 (t, 2H), 1.80 (m, 2H), 1.40 (m, 6H), 0.90 (m, 3H).

### Preparation of the intermediate methyl-5-[(3-hexyloxy)phenoxyl]pentanoate

The titled compound was prepared starting from 3-hexyloxyphenol (prepared as above described), (360 mg, 1.85 mmol) in anhydrous DMF (14.4.mL) and NaH 80% (61.5 mg, 2.03 mmol). After one hour, methyl 5-bromovalerate (361 mg, 1.85 mmol) was added, the reaction mixture was left under magnetic stirring at 60° C. for 18 hours, then H₂O (100 mL) was added and the mixture was extracted with AcOEt (3×30 mL). The combined organic layers were washed with water, dried on Na₂SO₄ and evaporated under vacuum. The residue was purified by two chromatographies on silica gel using in the first hexane/AcOEt 97/3, in the second CH₂Cl₂/hexane 80/20 and 85/15, to give 408 mg of an oily product (70% yield); ¹H NMR (CDCl₃, 300 MHz), δ 7.10 (t, 1H), 6.50 (m, 3H), 3.98 (m, 4H), 3.70 (s, 3H), 2.40 (brt, 2H), 1.98 (m, 6H), 1.40 (m, 6H), 0.90 (m, 3H).

### Preparation of the intermediate acid 5-[(3-hexyloxy)phenoxyl]pentanoic

To a solution of methyl 5-[3-(hexyloxy)phenoxy]pentanoate, (3.4 g, 11.02 mmol) in 216 mL of CH₃OH, were added NaOH 2N (11.05 mL) and H₂O (59 mL) and the reaction mixture was warmed up to 50° C. for 3 hours and then left at room temperature for other 18 hours. The solution was then evaporated under vacuum and the residue diluted with H₂O and extracted with AcOEt. The basic aqueous phase was acidified to pH 2 with HCl 2N, and extracted with AcOEt (3×250 mL). The combined organic phases were washed with water, dried on Na₂SO₄, filtered and then evaporated under vacuum to give 2.7 g of product (yield 83%) which was used without further purification; ¹H NMR (CDCl₃, 300 MHz), δ 7.20 (t, 1H), 6.50 (m, 3H), 3.98 (m, 4H), 2.50 (m, 2H), 1.85 (m, 6H), 1.40 (m, 6H), 0.95 (m, 3H).

### Preraration of (R)-4-trimethylammonium-3-[[4-[(3-hexyloxy)-phenoxy]butyl]carbamoyl]-amino-butyrate (ST2425)

To a solution of acid 5-[(3-hexyloxy)phenoxy]pentanoic, (1.3 g, 4.41 mmol) in CH₂Cl₂ (6.5 mL), CO₂Cl₂ (3.4 g, 26.4 mmol) was added a 0° C. and the reaction was left at 10° C. for 2 hours under magnetic stirring. The organic solvent was then evaporated under vacuum and the residue was washed three times with anhydrous diethilic ether. The oily residue was used without further purification. NaN₃ (488 mg, 7.50 mmol) was dissolved in H₂O (1.7 mL) and the solution so obtained was cooled to 8-15° C.: To this solution the acyl chloride above prepared dissolved in 1.7 mL of acetone was added. The reaction was left for ten minutes at this range of temperature and for an additional hour at room temperature. After this time the reaction was poured in a flask with toluene (5.5 mL), and the solution was heated at 70° C. under magnetic stirring. The organic layer was evaporated under vacuum and the residue obtained was used without further purification.

The obtained isocyanate was added to (R)-aminocarnitine (706 mg, 4.41 mmol) dissolved in anhydrous CH₃OH (53 mL) at 5° C. and the reaction was left for 18 hours at room temperature under magnetic stirring. The reaction mixture was then evaporated under vacuum and the residue purified by silica gel chromatography using as eluent CH₃OH/CHCl₃ from 7/3 to 8/2 to give 370 mg of a white solid (18.6%, yield). TLC: silica gel R_{f}=0.59, eluent CHCl₃:MeOH:isopropanol:CH₃COOH:H₂O 42:28:7:10.5:10.5; ¹H NMR (MeOH_{d4}, 300 MHz) δ 7.10 (t, 1H), 6.45 (m, 3H), 4.50 (brm, 1H), 3.90 (q, 4H), 3.50 (m, 2H), 3.20 (s, 9H), 2.40 (m, 2H), 1.75 (m, 6H), 1.45 (m, 6H), 1.20 (m, 2H), 0.90 (t, 3H); HPLC: column Symmetry-C18 (5 μm) 150×4.6 mm, mobile phase CH₃CN/NH₄H₂PO₄ 50 mM (40/60 v/v), pH as it is, room temperature, flow rate=1.0 mL/min, detector UV 205 nm, retention time=5.8 min; [α]²⁰_{D}=−15° (c=0.2% MeOH); KF=3.2% H₂O; A.E. conforms for C₂₄H₄₁N₃O₅.

### Example 5

### Preparation of (R)-4-trimethylphosphonium-3-[[4-[(3-hexyloxy)phenoxy]butyl]carbamoyl]-amino-butyrate (ST2452)

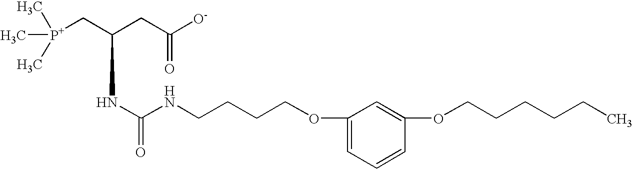

To the 4-[(3-hexyloxy)phenoxy]butyl]isocyanate, obtained as described in example 4 (ST2425), dissolved in CH₃OH (20 mL) and cooled at 5° C. (R)-phosphoaminocarnitine was added (781 mg, 4.41 mmol) dissolved in CH₃OH (38 mL). The reaction mixture was left under magnetic stirring for 72 hours then the solvent was evaporated and the residue purified with silica gel chromatography eluting with CHCl₃/CH₃OH from 7/3 to 8/2, to give 600 mg of product (23% yield); TLC: silica gel R_{f}=0.55, CHCl₃: iPrOH: MeOH: H₂O: CH₃COOH (42:7:28:10.5:10.5); [α]²⁰_{D}=−14.4°, c=0.5% MeOH; ¹H NMR (MeOH d4, 300 MHz): δ 7.15 (t, 1H), 6.45 (m, 3H), 4.40 (m, 1H), 3.95 (q, 4H), 3.20 (t, 2H), 2.70-2.40 (m, 4H), 1.90-1.30 (m, 21H), 0.90 (t, 3H); HPLC: column Symmetry C18 (5 μm), 4.6×150 mm, T=30° C., mobile phase CH₃CN/NH₄H₂PO₄ 50 mM (35/65 v/v) pH=as it is, flow rate=1 mL/min, detectors ═RI, UV 205 nm, retention time=10.1 min; A.E. conforms for C₂₄H₄₁N₂O₅P; KF=2.2% H₂O.

### Example 6

### Preparation of (R)-4-trimethylammonium-3-[[4-[(2-hexyloxy)-phenoxy]butyl]carbamoyl]-amino-butyrate (ST4005)

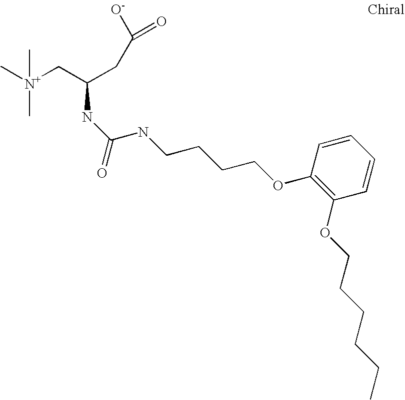

### Preparation of the intermediate methyl-5-[(2-hexyloxy)phenoxy]butyrate

The titled compound was prepared starting from 2-hexyloxyphenol (prepared as described in example 4 for 3-hexyloxyphenol), (750 mg, 3.82 mmol) in anhydrous CH₃CN (60 mL) and KOH (256 mg, 4.58 mmol). After one hour, methyl 5-bromovalerate (0.745 mg, 3.82 mmol) was added, the reaction mixture was left under magnetic stirring at 60° C. for 48 hours. The reaction mixture was evaporated under vacuum, then H₂O (100 mL) was added and the mixture was extracted with AcOEt (3×30 mL). The combined organic layers were washed with water, dried on Na₂SO₄ and evaporated under vacuum to give 705 mg of oil product (yield 60%).

¹H NMR (CDCl₃, 300 MHz), δ 6.9 (m, 4H), 4.00 (m, 4H), 3.70 (s, 3H), 3.40 (t, 2H), 2.40 (m, 4H), 1.90 (m, 8H), 0.90 (m, 3H).

### Preparation of the intermediate acid 5-[(2-hexyloxy)phenoxy]butyric

To a solution of methyl 5-[2-(hexyloxy)phenoxy]butyrate (1.8 g, 5.79 mmol) in 100 mL of CH₃OH, were added NaOH 2N (22 mL) and H₂O (29 mL) and the reaction mixture was warmed up to 50° C. for 3 hours. The solution was then evaporated under vacuum and the residue diluted with H₂O and extracted with AcOEt. The basic aqueous phase was acidified to pH 2 with HCl 2N, and extracted with AcOEt (3×250 mL). The combined organic phases were washed with water, dried on Na₂SO₄, filtered and then evaporated under vacuum to give 940 mg of product (yield 55%) which was used without further purification; ¹H NMR (CDCl₃, 300 MHz), δ 6.90 (m, 4H), 4.00 (m, 4H), 2.5 (t, 2H), 1.90 (m, 6H), 1.20 (m, 6H) 0.95 (m, 3H).

### Preparation of (R)-4-trimethylammonium-3-[[4-[(2-hexyloxy)-phenoxy]butyl]carbamoyl]-amino-butyrate (ST4005)

To a solution of acid 5-[(2-hexyloxy)phenoxy]butanoic, (500 mg, 1.68 mmol) in THF dry (8.7 mL), TEA (170 mg, 1.68 mmol), diphenyl phosphoryl azide (463 mg, 1.68 mmol) were added a 0° C. and the reaction was left at 80° C. for 18 hours under magnetic stirring.

After this time R-aminocarnitine (240 mg, 1.5 mmol) was added dissolved in MeOH dry (12.4 mL) to 5-10° C., then the reaction mixture was left at room temperature under magnetic stirring for 18 hours. The reaction mixture was then evaporated under vacuum and the residue purified by silica gel chromatography using as eluent CH₃OH/AcOEt from 7/3 to 8/2 to give 310 mg of a white solid (48%, yield). TLC: silica gel R_{f}=0.56, eluent CHCl₃:MeOH:isopropanol:CH₃COOH:H₂O 42:28:7:10.5:10.5; ¹H NMR (MeOHd₄, 300 MHz) δ 6.90 (m, 4H), 4.50 (brm, 1H), 4.00 (q, 4H), 3.50 (m, 2H), 3.20 (m, 11H), 2.40 (m, 2H), 1.85 (m, 6H), 1.45 (m, 6H), 0.90 (t, 3H); ESI-MS [M+H⁺] 452.2; [M+Na⁺] 474.2

### Example 7

### Preparation of (R)-4-trimethylammonium-3-[[4-[(3-hexyloxy)-phenoxy]propil]carbamoyl]-amino-butyrate (ST4024)

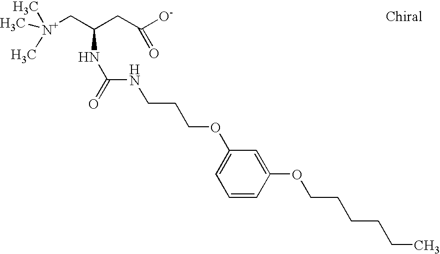

### Preparation of the intermediate methyl-5-[(3-hexyloxy)phenoxy]butyrate

The titled compound was prepared starting from 3-hexyloxyphenol (prepared as described in example 4), (1 g, 5.47 mmol) in anhydrous CH₃CN (80 mL) and K₂CO₃ (856 mg, 6.17 mmol). After one hour, methyl 4-bromobutanoate (1.8 g, 10.3 mmol) was added, the reaction mixture was left under magnetic stirring at 60° C. for 18 hours, then H₂O (100 mL) was added and the mixture was extracted with AcOEt (3×30 mL). The combined organic layer were washed with water, dried on Na₂SO₄ and evaporated under vacuum. The residue was purified by two chromatographies on silica gel using in the first hexane/AcOEt 98/2 to give 1.35 g of oil product (yield 66%). ¹H NMR (CDCl₃, 300 MHz), δ 7.20 (t, 1H), 6.50 (m, 3H), 3.98 (dt, 4H), 3.65 (s, 3H), 2.60 (t, 2H), 2.1 (m, 2H), 1.90 (m, 2H), 1.4 (m, 6H), 0.95 (t, 3H).

### Preparation of the intermediate acid 5-[(3-hexyloxy)phenoxy]butyric

To a solution of methyl 4-[3-(hexyloxy)phenoxy]butyrate, (1.35 g, 4.58 mmol) in 80 mL of CH₃OH, were added NaOH 2N (17 mL) and H₂O (23 mL) and the reaction mixture was warmed up to 50° C. for 3 hours. The solution was then evaporated under vacuum and the residue diluted with H₂O and extracted with AcOEt. The basic aqueous phase was acidified to pH 2 with HCl 2N, and extracted with AcOEt (3×250 mL). The combined organic phases were washed with water, dried on Na₂SO₄, filtered and then evaporated under vacuum to give 1.2 g of product as white solid (yield 92%) which was used without further purification; ¹H NMR (CDCl₃, 300 MHz), δ 7.20 (t, 1H), 6.50 (m, 3H), 3.98 (dt, 4H), 2.60 (t, 2H), 2.1 (m, 2H), 1.90 (m, 2H), 1.4 (m, 6H), 0.95 (t, 3H).

### Preparation of (R)-4-trimethylammonium-3-[[4-[(3-hexyloxy)-phenoxy]propil]carbamoyl]-amino-butyrate (ST4024)

To a solution of acid 5-[(3-hexyloxy)phenoxy]butyric (1 g, 3.55 mmol) in THF dry (18.3 mL), TEA (0.359 mg, 3.55 mmol), diphenyl phosphoryl azide (976 mg, 3.55 mmol) was added a 0° C. and the reaction was left at 80° C. for 18 hours under magnetic stirring.

After this time R-aminocarnitine (506 mg, 3.16 mmol) was added dissolved in MeOH dry (12.4 mL) to 5-10° C., then the reaction mixture was left at room temperature under magnetic stirring for 18 hours. The reaction mixture was then evaporated under vacuum and the residue purified by silica gel chromatography using as eluent CH₃OH/AcOEt from 7/3 to 8/2 to give 635 mg of a white solid (46%, yield). TLC: silica gel R_{f}=0.57, eluent CHCl₃:MeOH: isopropanol:CH₃COOH:H₂O 42:28:7:10.5:10.5; ¹H NMR (MeOHd₄, 300 MHz) δ 7.10 (t, 1H), 6.45 (m, 3H), 4.50 (brm, 1H), 3.90 (m, 4H), 3.50 (m, 2H), 3.30 (m, 2H), 3.20 (s, 9H), 2.40 (m, 2H), 1.90 (m, 2H), 1.70 (m, 2H), 1.45 (m, 2H), 1.30 (m, 4H), 0.90 (t, 3H); ESI-MS [M+Na⁺] 460.

### Example 8

### Preparation of (R)-4-trimethylammonio-3-[[3-(hexyloxy)phenoxy]acetyl]-amino-butyrate (ST4004)

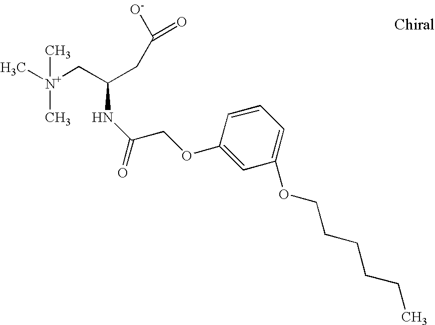

### Preparation of the intermediate ethyl-2-[(3-hexyloxy)phenoxy]acetate

The titled compound was prepared starting from 3-hexyloxyphenol (prepared as described in example 4), (1 g, 5.47 mmol) in anhydrous CH₃CN (80 mL) and K₂CO₃ (853 mg, 6.1 mmol). After one hour, ethyl-2-bromoacetate (1.14 mL, 1.7 g, 10.3 mmol) was added, the reaction mixture was left under magnetic stirring at 60° C. for 18 hours. The reaction mixture was evaporated under vacuum after filtration to give 1.4 g of oil compound, which was used without further purification.

¹H NMR (CDCl₃, 300 MHz), δ 7.20 (t, 1H), 6.50 (m, 3H), 4.65 (s, 2H), 4.25 (q, 2H), 3.98 (t, 2H), 1.80 (m, 2H), 1.50 (m, 2H), 1.3 (m, 7H), 0.95 (m, 3H).

### Preparation of the intermediate acid 2-[(3-hexyloxy)phenoxy]acetic

To a solution of ethyl 2-[3-(hexyloxy)phenoxy]acetate, (1.25 g, 4.46 mmol) in 78 mL of ethanol, were added NaOH 2N (15 mL) and H₂O (22 mL) and the reaction mixture was warmed up to 50° C. for 3 hours. The solution was then evaporated under vacuum and the residue diluted with H₂O and extracted with AcOEt. The basic aqueous phase was acidified to pH 2 with HCl 2N, and extracted with AcOEt (3×250 mL). The combined organic phases were washed with water, dried on Na₂SO₄, filtered and then evaporated under vacuum to give 1 g of product (yield 89%) which was used without further purification. ¹H NMR (CDCl₃, 300 MHz), δ 7.20 (t, 1H), 6.50 (m, 3H), 4.65 (s, 2H), 3.98 (t, 2H), 1.80 (m, 2H), 1.50 (m, 2H), 1.3 (m, 4H), 0.95 (m, 3H).

### Preparation of (R)-4-trimethylammonio-3-[[3-(hexyloxy)phenoxy]acetyl]-amino-butyrate (ST4004)

To a solution of acid 2-[(3-hexyloxy)phenoxy]acetic, (400 mg, 1.58 mmol) in CH₂Cl₂ dry (6 mL), 1-chloro-2-N,N-trimethyl-1-propenylamine (255 mg, 1.9 mmol) was added a 0° C. and the reaction was left at room temperature for 3 hours under magnetic stirring. The organic solvent was then evaporated under vacuum and the residue was washed three times with anhydrous diethyl ether. The compound was used without further purification and was dissolved in CH₂Cl₂ dry (1 mL) and added dropwise to R-4-trimetilammonio-3-amino-butyrrate (203 mg, 1.27 mmol) in MeOH dry (8 mL). The reaction was left at room temperature under magnetic stirring for 18 h.

The reaction mixture was then evaporated under vacuum and the residue purified by silica gel chromatography using as eluent CH₃OH/AcOEt from 7/3 to 9/1 to give 106 mg of compound (22%, yield). TLC: silica gel Rf=0.54, eluent CHCl₃:MeOH:isopropanol:CH₃COOH:H₂O 42:28:7:10.5:10.5; ¹H NMR (MeOH_{d4}, 300 MHz) δ 7.10 (t, 1H), 6.60 (m, 3H), 4.80 (brm, 1H), 4.60 (s, 2H), 4.00 (m, 2H), 3.60 (m, 2H), 3.20 (s, 9H), 2.50 (dq, 2H), 1.80 (m, 2H), 1.50 (m, 2H), 1.40 (m, 4H), 0.90 (t, 3H); ESI-MS [M+Na⁺] 417.

### Biological Studies

### In Vitro Inhibition of CPT I

The inhibition of CPT was evaluated on fresh mitochondrial preparations obtained from the liver or heart of Fischer rats, fed normally; the mitochondria taken from the liver or heart are suspended in a 75 mM sucrose buffer, EGTA 1 mM, pH 7.5. 100 μl of a mitochondrial suspension, containing 50 μM of [¹⁴C] palmitoyl-CoA (spec.act. 10000 dpm/mole) and 10 mM of L-carnitine, are incubated at 37° C. in the presence of stepped concentrations (0-3 mM) of product under examination.

Reaction time: 1 minute.

The IC₅₀ is then determined. The results are reported in Table 1.

TABLE 1
IC₅₀ of the inhibition curve of CPT1 in rat mitochondria

| Compound | Structure | IC₅₀ (heart) | IC₅₀ (liver) | Ratio |
|---|---|---|---|---|
| ST1326 | 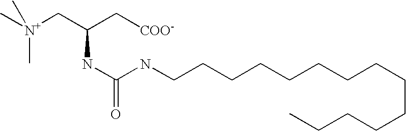 | 48.8 μM | 0.36 μM | 135 |
| ST2425 | 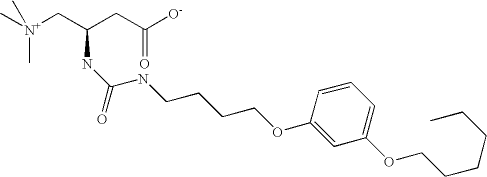 | 31.6 μM | 0.27 μM | 117 |
| ST2452 | 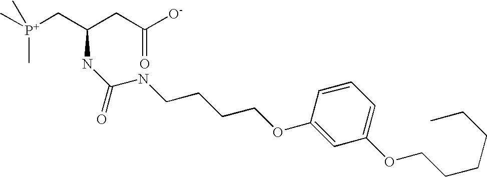 | 57.3 μM | 0.12 μM | 478 |

Inhibition of Ketone Bodies Production In Vivo by ST2425 and ST2452 in Comparison with ST1326

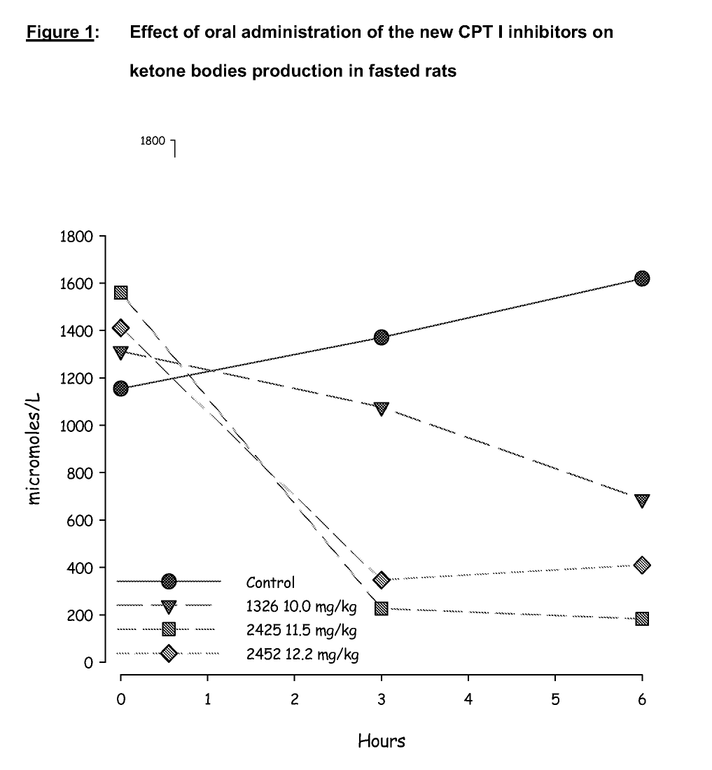

The inhibition of CPT and consequently of β-hydroxybutyrate production operated by ST2425 and ST2452 was evaluated in vivo in rats fasted from 17 hours, at doses equimolar to 10 mg/Kg of ST1326 used as reference compound. β-hydroxybutyrate levels were measured at 3 and 6 hours from single oral treatment. As shown in FIG. 1 with ST2425 and ST2452, the reduction of β-hydroxybutyrate levels was higher and faster with respect to ST1326, reaching after 3 hours the minimum values, that were maintained stable for additional 3 hours.

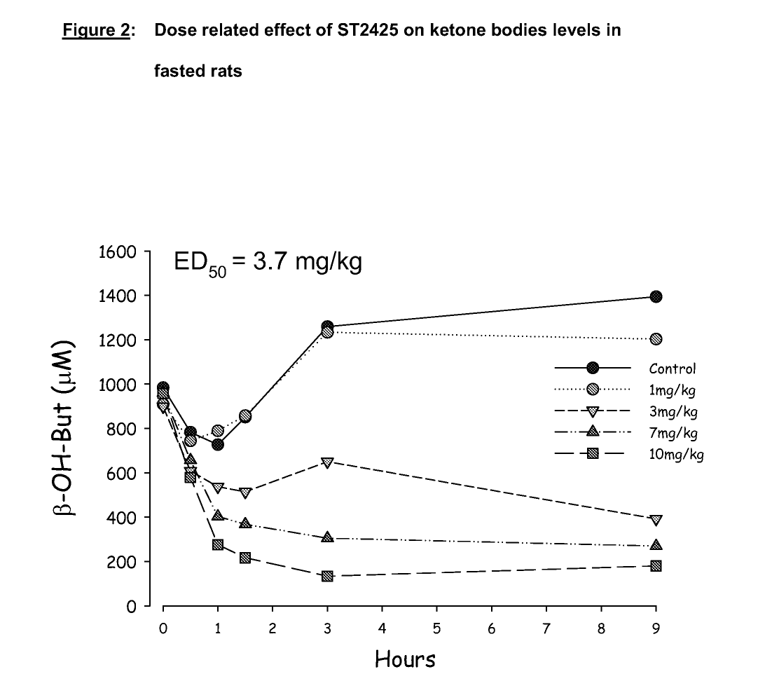

For compound ST2425, inhibition of ketone bodies production was also evaluated at the dosages of 0, 1, 3, 7, 10 mg/kg in rats fasted for 16 hours, following the reduction of β-hydroxybutyrate for 9 hours after single oral treatment. ED₅₀ value, calculated on the basis of AUC from time 0 to 9 hours, was equal to 3.7 mg/kg, lower than that found for ST1326 (ED₅₀=14.5 mg/kg). As shown on FIG. 2 a faster onset of action was also observed.

Antihyperglycemic Activity of ST2425 and ST2452 in db/db Mice

ST2425 and ST2452 were administered to db/db mice for 12 days at 30 mg/kg/day, using ST1326 at a higher dose (80 mg/kg/day) as reference compound. At the end of the treatment, serum glucose levels were evaluated after 16 hours fasting and 2 hours from last administration. The results are reported in Table 2, which shows that ST2425 induced a 41% and ST2452 a 30% reduction of glucose levels, while with ST1326 a 26% reduction was observed in spite of the almost 3 times higher dosage.

### Table 3 Anti-hyperglycemic Activity

TABLE 2
Anti-hyperglycemic Activity

| | | Glucose |
|---|---|---|
| Compounds | Dosage | (mg/dL) |
| Control | Vehicle | 709 ± 79 |
| ST1326 | 80 mg/kg | 521* ± 131 |
| ST2425 | 30 mg/kg | 418* ± 114 |
| ST2452 | 30 mg/kg | 492* ± 108 |

Mean ± SD (n=7);
*= p < 0.05 vs control, Student's t test.

Mean±SD (n=7); *=p<0.05 vs control, Student's t test.

### Effect on Food Intake and Body Weight of Repeated Intracerebroventricular Administration of ST2425

Two groups of 8 C57BL/6J mice each (ST2425 and Control) were injected icv (3 μL) with 250 pmoles (0.113 μg) of ST2425 dissolved in RPMI 1640 (vehicle), for 4 days (starting from day 0). The animals were slightly confused by isofluorane anestesia. The head was positioned in an apparatus used to reveal a “virtual bregma” without opening the skin. The injection was performed with a syringe at 3.5 mm of depth, using the following coordinates from Bregma: 1 mm on the left of the midsagittal suture and 3 mm posterior (lateral ventricle). The animals were treated at 5:00 p.m. and food intake was monitored at 8:00 a.m. of the day after. Starting from the day after the first treatment, food was removed from 8:00 a.m. to 5:00 p.m.

Statistical analysis was performed using two way repeated measures ANOVA followed by Student, Newman, Keuls test as post-hoc analysis.

The results are reported in Tables 3 and 4 and show that ST2425 reduced food consumption (−25%) and mice weight (−7%) with respect to the control group over the experiment. A statistically significant reduction of food intake was observed on days 3 and 4 (about 30%), and of mice weight on days 2 (−5%), 3 and 4 (−10%).

TABLE 3
Effect of repeated intracerebroventricular administration
of ST2425 on food intake of C57BL6/J mice.

| | | 24 hours food intake (g) | |
|---|---|---|---|
| | Day of | Mean ± S.D. | |
| Species/Strain/number/sex | experiment | Control | ST2425 |
| Mouse/C57BL6J/8/male | 0 | — | — |
| | 1 | 3.67 ± 0.49 | 3.15 ± 0.61 |
| | 2 | 3.93 ± 0.45 | 3.31 ± 0.70 |
| | 3 | 5.13 ± 0.46 | 3.35 ± 0.73* |
| | 4 | 5.14 ± 0.46 | 3.55 ± 0.63* |

8 animals for each group.

Two Way Repeated Measures ANOVA, group, F_{(1, 14)}=41.4, p<0.001; time, F_{(3, 42)}=14.9, p<0.001; group x time, F_{(3, 63)}=7.32, p<0.001. Post-Hoc analysis, comparison for factor treatment: *=p<0.05 vs. control.

TABLE 4
Effect of repeated intracerebroventricular
administration of ST2425 on mice weight.

| | | Mice weight (g) | |
|---|---|---|---|
| | Day of | Mean ± S.D. | |
| Species/Strain/number/sex | experiment | Control | ST2425 |
| Mouse/C57BL6J/8/male | 0 | 21.7 ± 1.23 | 22.2 ± 0.91 |
| | 1 | 21.7 ± 0.95 | 21.2 ± 0.96 |
| | 2 | 21.7 ± 0.72 | 20.5 ± 1.14* |
| | 3 | 22.4 ± 0.75 | 20.0 ± 0.69* |
| | 4 | 22.6 ± 0.66 | 20.1 ± 0.64* |

8 animals for each group.

Two Way Repeated Measures ANOVA, group, F_{(1, 14)}=22.1, p<0.001; time, F_{(3, 42)}=1.8, ns; group x time, F_{(3, 63)}=12.6, p<0.001. Post-Hoc analysis, comparison for factor treatment: *=p<0.05 vs. control.

### Effect on Food Intake of Intranasal Administration of ST2425 in Normal Rats

To test the activity on food consumption of the compounds of the present invention after intranasal administration, ST2425 was given to normally-fed Sprague Dawley rats (320 μg/40 μL/rat, in citrate buffer 10 mmol/L pH 5.0, equally subdivided into the two nostrils) 2 h before the dark cycle. The compound was administered for 3 days (starting from day 0) and food consumption was measured every time for the following 24 h. Five rats were considered for each group.

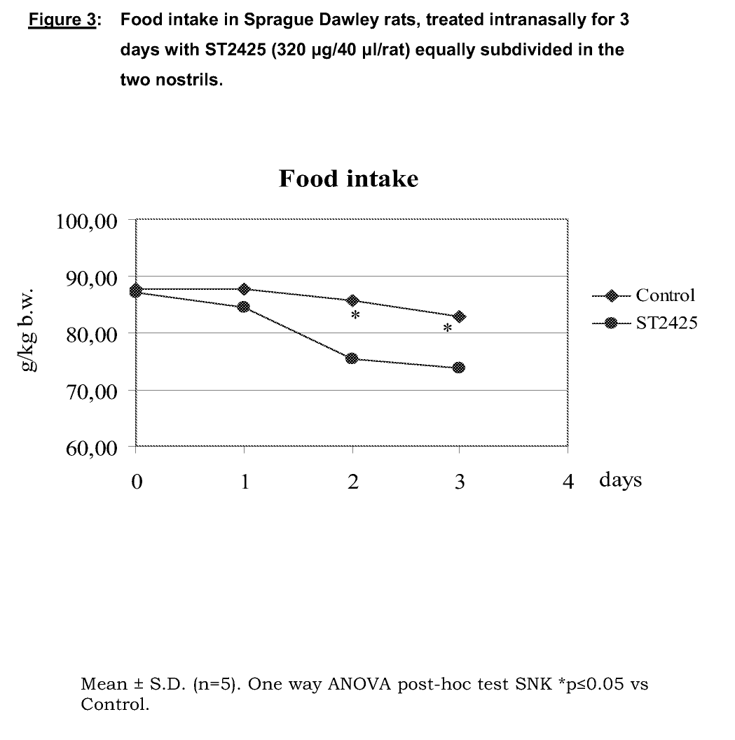

A significant reduction of food consumption was observed with respect to controls starting from the day after the second treatment with ST2425, as shown in FIG. 3.

1. A compound having the following Formula (I): 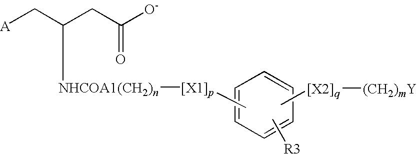
wherein:
A is —N⁺ (R R₁ R₂), or —P⁺ (R R₁ R₂), in which R R₁, R₂ are the same or different and are selected from the group consisting of (C₁-C₂) alkyl, phenyl and phenyl-(C₁-C₂) alkyl;
A1 is O or NH or is absent;
n is an integer ranging from 0 to 20;
p is 0 or 1;
q is O or 1;
X1 is O or S;
X2 is O or S;
m is an integer ranging from 1 to 20;
Y selected among H, phenyl and phenoxy;
R3 is selected among H, halogen, linear or branched (C₁-C₄) alkyl and (C₁-C₄) alkoxy or a pharmaceutically acceptable salt thereof.

2. The compound of claim 1 wherein R, R₁ and R₂ are all methyl.

3. A compound of claim 1, which is selected from the group consisting of:
(R)-4-trimethylammonium-3-[[4-[(3-hexyloxy)-phenoxy]butyl]carbamoyl]-amino-butyrate;
(R)-4-trimethylphosphonium-3-[[4-[(3-hexyloxy)-phenoxy]butyl]carbamoyl]-amino-butyrate;
(R)-4-trimethylammonium-3-[[4-(heptyloxy)-phenyl]-carbamoyl]-amino-butyrate;
(R)-4-trimethylammonium-3-[[2-(benzyloxy)-benzyl]carbamoyl]-amino-butyrate;
(R)-4-trimethylammonium-3-[[(4-benzyloxy-3-methoxy)-benzyl]carbamoyl]-amino-butyrate;
(R)-4-trimethylammonium-3-[[4-[(2-hexyloxy)-phenoxy]butyl]carbamoyl]-amino-butyrate;
(R)-4-trimethylammonium-3-[[4-[(3-hexyloxy)-phenoxy]propil]carbamoyl]-amino-butyrate; and
(R)-4-trimethylammonio-3-[[3-(hexyloxy)phenoxy]acetyl]-amino-butyrate.

4. A compound of claim 1, as a medicament.

5. A process for preparing a compound of claim 1, comprising reacting aminocarnitine and phosphoaminocarnitine with the corresponding isocyanates, in a dipolar aprotic or protic solvent at a temperature between 4° C. and the reflux temperature of the solvent for between 1 and 72 hours.

6.-11. (canceled)

11. A pharmaceutical composition containing as active ingredient a compound of claim 1, and at least one pharmaceutically acceptable excipient and/or diluent.

12. (canceled)

13. A process for the preparation of the pharmaceutical composition of claim 11, comprising mixing a compound of claim 1 with at least one pharmaceutically acceptable excipient and/or diluent.

14. (canceled)

15. A method of treating or preventing a condition selected from the group consisting of obesity, hyperglycaemia, diabetes and diabetes-associated disorders, in a mammal, comprising administering a therapeutically effective amount of a compound of claim 1 to a mammal in need thereof.

16. The method of claim 15, wherein the diabetes-associated disorder is diabetic retinopathy, diabetic neuropathy or a cardiovascular disorder.

17. A method of treating or preventing cardiac disorders, in a mammal, comprising administering a therapeutically effective amount of a compound of claim 1 to a mammal in need thereof.

18. The method of claim 17, wherein the cardiac disorder is congestive heart failure.
